(19) United States
(12) Patent Application Publication
(10) Pub. No.: US 2024/0329047 A1
(43) Pub. Date: Oct. 3, 2024

(54) CANCER RISK ASSESSMENT

(71) Applicant: Micromass UK Limited, Wilmslow (GB)

(72) Inventors: Johannes Vissers, Breda (NL); Billy J. Molloy, Stockport (GB); Dominic Foley, Barrow-in-Furness (GB)

(73) Assignee: Micromass UK Limited, Wilmslow (GB)

(21) Appl. No.: 18/619,589

(22) Filed: Mar. 28, 2024

Related U.S. Application Data
(60) Provisional application No. 63/455,794, filed on Mar. 30, 2023.

(51) Int. Cl. G01N 33/574; G01N 33/68

(52) U.S. Cl. CPC G01N 33/57476; G01N 33/57484; G01N 33/6851; G01N 2333/976; G01N 2333/98

(57) ABSTRACT

The present disclosure relates to methods of quantifying a target protein e.g., a tumor biomarker, in a sample by using a mass spectroscopy coupled with a chromatography method. The methods presented herein include incubating the sample with a composition comprising an amidase and a protease for a predetermined amount of time. Incubating the sample with a composition comprising an amidase and a protease in a single step improves the digestion efficiency of the target protein, leading to enhanced detectability e.g., signal to noise ratio, in mass spectroscopy.

### CROSS-REFERENCE TO RELATED APPLICATIONS

This application claims priority to U.S. Provisional Patent Application No. 63/455,794 filed on Mar. 30, 2023, the entirety of which is incorporated by reference herein.

### FIELD OF THE TECHNOLOGY

The present disclosure discusses methods for quantifying a target protein e.g., a tumor biomarker, in a sample by using LC-MS analysis. The methods disclosed herein include improved digestion protocols, i.e., treating the sample with a composition including an amidase and a protease for a predetermined amount of time prior to LC-MS analysis.

### BACKGROUND

In quantitative analysis of proteins, e.g., cancer biomarkers and therapeutic proteins, by liquid chromatography/mass spectrometry (LC-MS), digesting the proteins with proteolytic enzymes to produce smaller peptide fragments prior to LC-MS is a well-established approach. In-solution digestion is one widely used method for protein digestion.

Post-translational modification of proteins with glycosylation is of key importance in many biological systems in eukaryotes, influencing fundamental biological processes and regulating protein function. Glycosylation is the reaction in which a carbohydrate (or ‘glycan’), i.e., a glycosyl donor, is attached to a hydroxyl or other functional group of a target macromolecule in order to form a glycoconjugate. However, MS-based characterization of glycoproteins following proteolytic cleavage can be challenging due to the structural variations or steric hindrance imposed by the attached glycans or any other form of structural interference limiting protease surface access. The presence of glycosylation can inhibit protease digestion and lower the quality and confidence of protein quantification/identification by mass spectrometry.

While deglycosylation prior to protease digestion can improve the efficiency of subsequent protease digest, this step is often excluded from LC-MS workflows since it suffers from multiple drawbacks e.g., extended hours of incubation with a protease. Alternatives to conventional digestion protocols, that increase the rate of enzymatic cleavage of glycoproteins, may aid in addressing these challenges.

### SUMMARY

The problems encountered in prior art can be overcome using one-step digestion protocol that applies both an amidase and a protease together for a predetermined amount of time to enzymatically digest a target protein, e.g., a glycoprotein. That is, enzymatic digestion of a glycoprotein can be accelerated and made more efficient through one-step digestion protocol using both an amidase and a protease concurrently. The glycoprotein may be selected from a cancer biomarker or therapeutic proteins.

LC-MS based quantitative analysis of proteins could benefit from improved digestion protocols disclosed herein, which can subsequently lead to enhanced quantitative performance in terms of signal to noise ratio, limit of detection (LOD), upper and lower limit of quantification (ULOQ and LLOQ), dynamic range, accuracy, precision, etc.

The methods outlined in the present disclosure for enzymatic (e.g., proteolytic) digestion of proteins are compatible with LC-MS procedures, along with procedures that allow for simplification of complex protein matrices (e.g., denaturing protocols).

In one aspect, provided herein is a method of quantifying a target protein in a sample. The method includes the steps of: incubating the sample with a composition including an amidase and a protease for a predetermined amount of time; subjecting the sample to a chromatography method; and quantifying the target protein using a mass spectroscopy method.

In some embodiments, the protease is active within a first pH range, e.g., 4.0-8.0. In some embodiments, the amidase is active within a second pH range, e.g., 4.0-9.5. In some embodiments, a pH of the composition including an amidase and a protease is within a range that the first pH range and the second pH range overlaps.

In some embodiments, the protease is trypsin, Lys-C, Lys-N, Arg-C, Glu-C, Asp-N, chymotrypsin, or combinations thereof. In some embodiments, the amidase is PNGaseF, PNGaseA, Endoglycosidase H, Endoglycosidase F, and Exoglycosidase. In some embodiments, the protease is trypsin and the amidase is PNGaseF.

In some embodiments, the target protein is a glycoprotein selected from a cancer biomarker. In some embodiments, the target protein is cancer antigen 125 (CA-125) or carcinoembryonic antigen (CEA).

In some embodiments, the mass spectrometry method includes a mass spectroscopy selected from the group consisting of matrix-assisted laser desorption/ionization-time-of-flight (MALDI-TOF), electrospray-ionization (ESI), charge detection (CD), Fourier transform-ion cyclotron resonance (FT-ICR), ion-mobility spectrometry (IMS), triple or tandem quadrupole (QqQ), time of flight (TOF), and ion trap.

In some embodiments, the chromatography method includes a reverse phase separation, a cation exchange separation, an anion exchange separation, an ion pair separation, normal phase separation, an ion mobility separation, a size-exclusion separation, a chiral separation, an affinity separation, a ligand exchange separation, a polar nonionic separation, or any combination thereof.

In some embodiments, the chromatography method is coupled with an UV detection system.

In some embodiments, the chromatography method is coupled with a fluorescence detection system.

In some embodiments, the ratio of molar concentration of the protease to molar concentration of the target protein is between 1 and 100.

In some embodiments, the molar concentration of the protease is equivalent or substantially equivalent to the molar concentration of amidase. In certain embodiments the molar concentration of the protease is different (i.e., has a different molar concentration level) than the molar concentration of amidase.

In some embodiments, the predetermined amount of time is between about 2 hours to about 24 hours, between about 6 hours to about 24 hours, between about 10 hours to about 24 hours, between about 12 hours to about 24 hours, between about 16 hours to about 24 hours, between about 18 hours to about 24 hours.

In some embodiments, the predetermined amount of time is between about 5 minutes to 180 minutes, between about 10 minutes to 120 minutes, between about 10 minutes to 100 minutes, between about 10 minutes to 30 minutes, between about 15 minutes to 100 minutes, between about 15 minutes to 80 minutes, between about 15 minutes to 60 minutes, between about 15 minutes to 30 minutes.

In some embodiments, the method presented herein further comprises heating the sample for a predetermined amount of time prior to subjecting the sample to a chromatography method.

In some embodiments, the heating comprises maintaining the temperature in a range from about 30° C. to about 80° C., about 35° C. to about 70° C., 35° C. to about 50° C. for a predetermined amount of time.

In some embodiments, the method of the present technology further includes buffer exchange and/or desalting processes prior to quantifying the target protein using a mass spectroscopy method.

In some embodiments, the sample is or is derived from a biological fluid selected from the group consisting of blood, urine, spinal fluid, synovial fluid, sputum, semen, saliva, tears, gastric juices and extracts and/or dilutions/solutions thereof.

In one embodiment, the sample includes at least one target protein extracted from a biological fluid or a biological tissue.

In another aspect, provided herein is a method of quantifying a target protein in a sample. The method includes the steps of: incubating the sample with a composition including PNGaseF and trypsin for a predetermined amount of time; subjecting the sample to a chromatographic method; and quantifying the target protein using a mass spectrometry method. In some embodiments, the target protein is cancer antigen 125 (CA-125) or carcinoembryonic antigen (CEA).

In accordance with the multiple aspects and embodiments disclosed above, prior to incubating the sample with a composition including an amidase and a protease for a predetermined amount of time, the sample may need to be denatured, reduced and/or alkylated, using various reagents, for the proteolytic enzyme to be able to efficiently cleave the peptide chains of the proteins. In some embodiments, the various reagents that are used to denature, reduce and/or alkylate the sample prior to digestion may include at least one type of surfactant.

The methods of the present technology reveal time-saving and efficient digestion protocols for proteins prior to MS analysis. The one-step digestion protocol presented herein subsequently leads to improved detectability of proteins, e.g., a glycoprotein, in a sample.

### BRIEF DESCRIPTION OF THE DRAWINGS

The technology will be more fully understood from the following detailed description taken in conjunction with the accompanying drawings, in which:

FIG. 1 is a bar graph depicting the relative MS signal of detected peptides produced by incorporating deglycosylation at different stages prior to digestion, including the method in accordance with the multiple embodiments presented herein (e.g., trypsin and PNGaseF combined) for digesting carcinoembryonic antigen (CEA).

FIG. 2A shows detectable MS peaks (7 significant chromatographic peaks) that belong to peptides when trypsin and PNGaseF were used together to digest carcinoembryonic antigen (CEA) in accordance with the multiple embodiments of the present technology. FIG. 2B shows MS signal (no significant observable chromatographic peaks) when only trypsin was used for digestion of carcinoembryonic antigen (CEA).

FIG. 3A shows an MS chromatogram of increasing concentrations (up to 10,000 ng/ml) of cancer antigen 125 (CA-125) without PNGaseF treatment FIG. 3B shows an MS chromatogram of increasing concentrations (up to 1000 ng/ml) of cancer antigen 125 (CA-125) using PNGaseF for deglycosylation.

FIG. 4A shows an MS chromatogram of 500 ng/mL cancer antigen 125 (CA-125) in human plasma digested with trypsin without PNGaseF treatment FIG. 4B shows MS chromatogram of 500 ng/mL cancer antigen 125 (CA-125) in human plasma digested with trypsin with PNGaseF for deglycosylation.

### DETAILED DESCRIPTION

Protein analysis by LC-MS has already become an indispensable diagnostic tool to detect certain biomarker proteins that result in various diseases e.g., cancer. Yet, continual efforts have been made to improve the data quality from the LC-MS analysis, including using optimized experimental procedures, fine-tuned instrument parameters, as well as more advanced mass spectrometers. The present disclosure presents an efficient workflow including a novel protein digestion step to improve the LC-MS data quality, e.g., improved signal to noise ratio.

Proteins cannot always be directly analyzed by LC-MS, several sample preparation steps can be necessary. MS-based quantification and characterization of proteins has been achieved predominantly with the bottom-up approach, which typically involves the enzymatic cleavage of proteins to peptides prior to LC-MS or LC-MS/MS analysis. Glycosylation is an ubiquitous post-translational modification that plays a major role in the structural and functional properties of peptides and proteins. Therefore, various proteins (e.g., cancer antigen 125 (CA-125) in biological media possess glycans attached to their backbone structures. However, due to the structural variations and steric hindrance imposed by the attached glycans, which prevents or mitigates effective digestion of proteins, MS-based characterization of certain proteins can be challenging.

Conventional protocols involve deglycosylation techniques prior to protein digestion step to cause partial or complete cleavage of the glycan moieties attached to the peptide backbones. Conventional methods, however, often involve up to 12-24 hours of digestion times due to protein heterogeneity in the samples. Alternative methods e.g., heating, microspin columns, ultrasonic energy, high pressure, infrared (IR) energy, microwave energy, alternating electric fields and microreactors, have been introduced in order to speed up the digestion method.

Deglycosylation techniques are mostly utilized prior to qualitative (e.g., sequence mapping) analysis of proteins using LC-MS since to study the structure and function of a glycoprotein, it is often desirable to remove all or just a select class of glycans. This approach allows the assignment of specific biological functions to particular components of the glycoprotein. The removal of N-linked glycans from glycoproteins addresses heterogeneity in mass spectrometric (MS) analysis.

To overcome the problems of prior sample preparation methods, the present disclosure provides a method of quantifying a target protein in a sample, which includes, inter alia, a step of incubating the sample with a composition including an amidase and a protease for a predetermined amount of time. That is, deglycosylation and digestion of proteins occur in a single step. This single digestion step yields accelerated workflow for quantification of a target protein in a sample.

The present disclosure also shows that incubating the sample with an amidase and a protease in a single step improves digestion efficiency, and ultimately yields enhanced MS response e.g., higher signal-to-noise ratio. Obtaining higher signal to noise ratio may enable detection of low abundant proteins (e.g., cancer biomarkers) in a sample, which allows detection of diseases at early stages.

### Definitions

As used herein, the term “about” means that the numerical value is approximate and small variations would not significantly affect the practice of the disclosed embodiments. Where a numerical limitation is used, unless indicated otherwise by the context, “about” means the numerical value can vary by +10% and remain within the scope of the disclosed embodiments.

As used herein, the term “sample” refers to any medium that includes an analyte (e.g., a target nucleic acid or a target lipid molecule) to be processed using the compositions and methods according to the present disclosure. A sample may be selected from an agricultural sample, an environmental sample, or a biological sample. A biological sample may include, but is not limited to, for example, a formulated nucleic acid base drug, a formulated nucleic acid base vaccine, a clinical specimen (e.g., blood, plasma, serum, sputum, tissue, urine, saliva, sample/fluid from the respiratory tract, etc.), and cosmetic and pharmaceutical products (e.g., lotions, creams, ointments, solutions, medicines, eye and ear drops, etc.).

As used herein, the term “pretreating” refers to any steps or methods that treat a sample for downstream analysis of a target nucleic acid. Sample preparation may comprise various procedures needed to process the raw sample so that it is amenable to further analytical method, e.g., LC-MS method. It is important to note that at least one single sample preparation step should be compatible with downstream detection method in order to obtain optimal results.

As used herein, the term “predetermined amount of time” refers to a period of time in which a step of a method disclosed herein (e.g., incubation of a sample or heating of a sample) is performed. Said predetermined amount of time may be adjusted depending on particular experimental conditions as would be understood and evaluated by one of ordinary skill in the art.

As used herein, the term “protease” (also called a peptidase, proteinase, or proteolytic enzyme) is an enzyme that catalyzes (increases reaction rate or “speeds up”) proteolysis, breaking down proteins into smaller polypeptides or single amino acids. For example, trypsin is a serine proteases trypsin that catalyzes the hydrolysis of peptide bonds, breaking down proteins into smaller peptides. Trypsin cuts peptide chains mainly at the carboxyl side of the amino acids lysine (Lys, K) or arginine (Arg, R).

As used herein, the term “amidase” is an enzyme that catalyzes the hydrolysis of an amide, thereby resulting in the cleavage of an internal glycoside bond in an oligosaccharide. For example, PNGaseF catalyzes the cleavage of an internal glycoside bond in an oligosaccharide. It cleaves all asparagine (Asn or N)-linked complex, hybrid, or high mannose oligosaccharides unless the core GlcNAc contains an alpha 1,3-fucose. PNGaseF can be used to deglycosylate N-linked glycans on the protein in the sample.

The present technology provides an effective and accelerated workflow for quantifying a target protein in a sample by using LC-MS or LC-MS-MS. In one aspect, provided herein is a method of quantifying a target protein in a sample. The method includes incubating the sample with a composition comprising an amidase and a protease for a predetermined amount of time; subjecting the sample to a chromatography method; and quantifying the target protein using a mass spectroscopy method.

It is known in the art that the digestion efficiency of a protease is highly dependent on surface characteristics of a protein, e.g., structure, microenvironment, accessibility to cleavage sites. In the present methods, deglycosylation and digestion of proteins occur in a single step in which a sample including a target protein is incubated with an amidase and a protease. Without wishing to be bound by the theory, the observed increase in digestion efficiency of the proteins may be attributed to increased accessibility of proteases to cleavage sites due to removal of glycans on the surface of the proteins by the amidase.

In some examples, the amidase and the protease are in-solution. In other examples, the amidase and/or the protease are bound to a solid support.

In the present disclosure, pre-treatment of samples (including denaturation, reduction, and alkylation) can be conducted before digestion depending on the protein. In some examples, before protein digestion, there can be a pretreatment step. In some examples, proteins that can be easily denatured during digestion do not require pretreatment. For proteins that need pretreatment, denaturation followed with reduction and alkylation are common steps to fully unfold the protein. Desalting step may be needed prior to digestion or prior to chromatography method to remove reagents that can interfere with analysis of proteins.

The digestion conditions play important role for digestion efficiency. Digestion parameters like pH, temperature and digestion time may change depending on the target protein. In addition, a pH range chosen for the digestion needs to be suitable for both the amidase and the protease. In some embodiments, a pH of the composition including an amidase and a protease is between about pH 7 to about pH 8.

### EXAMPLES

### Example 1: Protein Quantification with a Combined Digestion Step

Trypsin is an enzyme that selectively cleaves proteins specifically at lysine and arginine. Peptide-N-glycosidase F (PNGaseF) is one of the most widely used enzymes for the deglycosylation of glycoproteins. The enzyme releases asparagine-linked (N-linked) oligosaccharides from glycoproteins and glycopeptides. The glycan can be a high-mannose, hybrid or complex type.

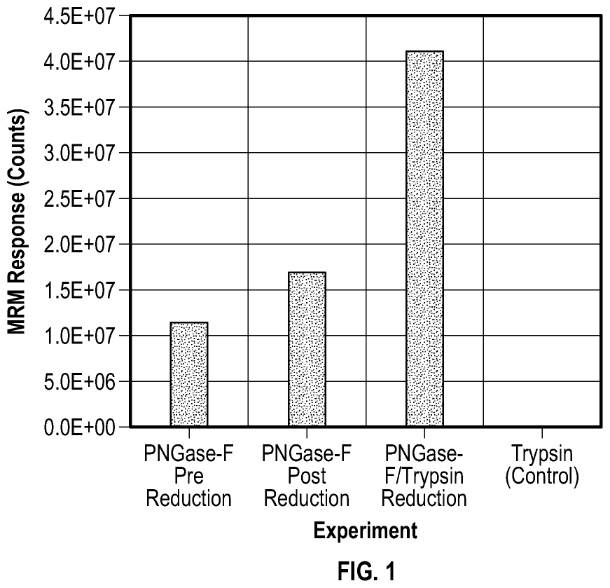

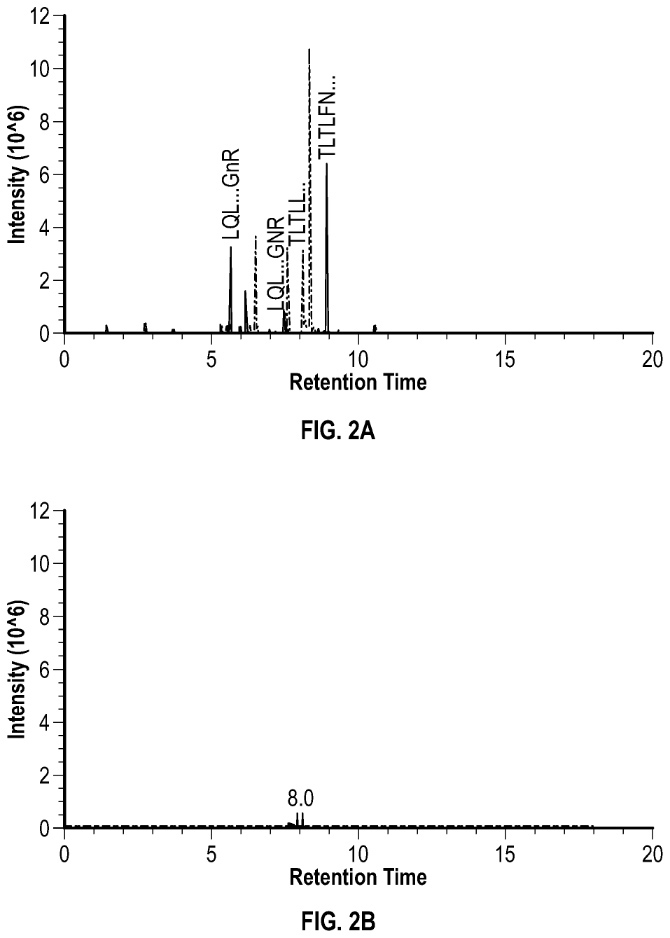

Optimal quantification of carcinoembryonic antigen (CEA) by using multiple reaction monitoring mass spectrometry (MRM MS) was achieved with PNGaseF deglycosylation and complementary proteolysis with trypsin. This combination of deglycosylation and complementary protease digest that occurred in single step yielded increased digestion efficiency (ultimately increased signal to noise ratio in MS) (FIG. 1) and the number of detectable surrogate candidate peptides (FIG. 2A). The surrogate candidate peptides used herein refer to the peptides generated when the target protein is digested. The surrogate peptide approach (FIG. 1, FIG. 2A and FIG. 2B) is an indirect approach, in which the target protein is digested to smaller peptides first and then the generated peptides are analyzed as surrogate analytes of the target protein.

FIG. 1 shows the relative MS signal of detected peptides produced by different digestion methods. Each digestion method was performed overnight. The relative MS signals of the peptides was significantly greater when trypsin and PNGaseF are used concurrently in single digestion step, when compared to trypsin only digestion step. A pre or prior reduction method, PNGaseF-pre reduction method, was performed by the addition of PNGaseF prior to the reduction of the disulfide bridges of the protein (i.e., carcinoembryonic antigen (CEA)) while PNGaseF-post reduction method was performed by the addition of PNGaseF after the reduction of the disulfide bridges of the protein. FIG. 1 shows that incorporation deglycosylation within digestion step increases digestion efficiency leading to increased MS response.

Referring to FIG. 2A and FIG. 2B, the number of detectable peptide peaks increases greatly when trypsin and PNGaseF are used together (7 strong peaks), compared to when only trypsin is used (no significant signal observed).

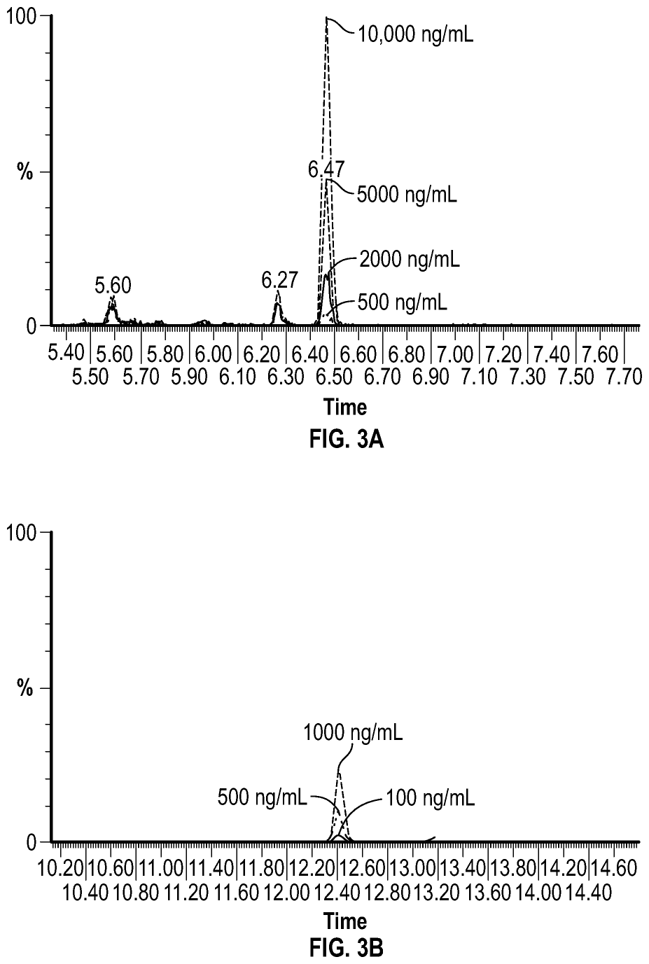

FIG. 3A shows an MS chromatogram of increasing concentrations (up to 10,000 ng/ml) of cancer antigen 125 (CA-125) without PNGascF. FIG. 3B shows an MS chromatogram of increasing concentrations (up to 1000 ng/ml) of cancer antigen 125 (CA-125) using PNGaseF for deglycosylation. FIG. 3A and FIG. 3B show that the deglycosylation does not have a negative effect on tryptic digestion of a moderately glycosylated protein. Further, the deglycosylation protocol used in this example yields better MS sensitivity for cancer antigen 125 (CA-125). For example, when the intensity of the peak corresponding to 500 ng/ml deglycosylated protein in FIG. 3B is compared with the intensity of the peak corresponding to 500 ng/ml protein without deglycosylation in FIG. 3A, there is a clear increase in the intensity of the peak in FIG. 3B. It is noted that the y-axis scale in FIG. 3B is comparable (i.e., substantially the same) as the y-axis scale of FIG. 3A.

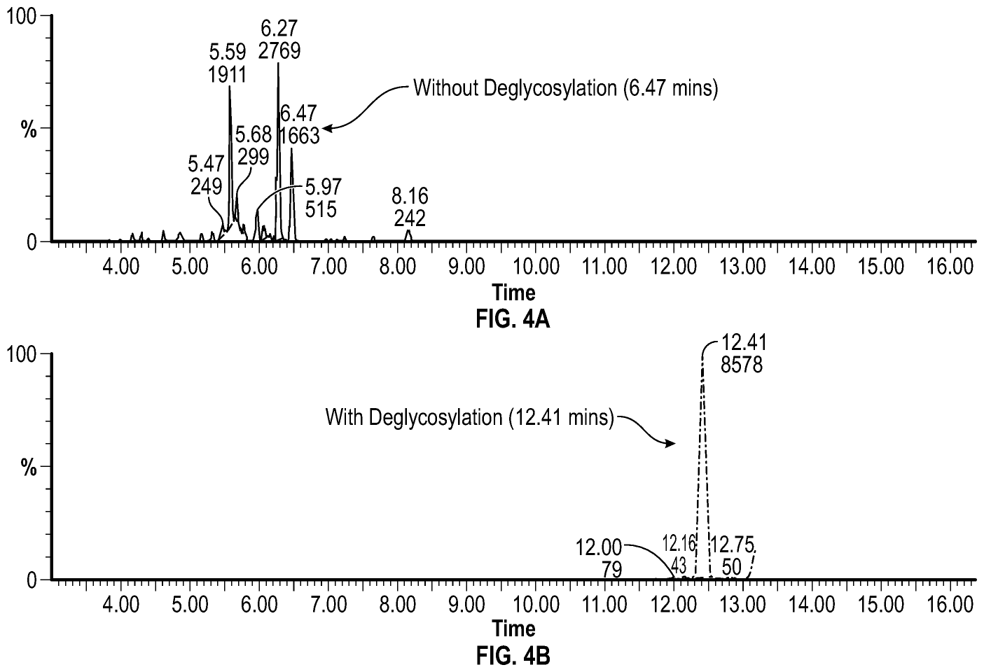

FIG. 4A shows an MS chromatogram of 500 ng/mL cancer antigen 125 (CA-125) in human plasma digested with trypsin without PNGaseF. FIG. 4B shows an MS chromatogram of 500 ng/mL cancer antigen 125 (CA-125) in human plasma digested with trypsin with PNGaseF. FIG. 4A and FIG. 4B present the same effect for CA-125 as FIG. 3A and FIG. 3B. However, in FIG. 4A and FIG. 4B, the gradient lengths are not equal than that of FIG. 3A and FIG. 3B (which accounts for the difference in retention time).

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A method of quantifying a target protein in a sample, the method comprising the steps of:
a. incubating the sample with a composition comprising an amidase and a protease for a predetermined amount of time;
b. subjecting the sample to a chromatography method; and
c. quantifying the target protein using a mass spectroscopy method.

2. The method of claim 1, wherein the protease is trypsin, Lys-C, Lys-N, Arg-C, Glu-C, Asp-N, chymotrypsin, or combinations thereof.

3. The method of claim 1, wherein the amidase is PNGaseF, PNGaseA, Endoglycosidase H, Endoglycosidase F, and Exoglycosidase.

4. The method of claim 1, wherein the protease is trypsin and the amidase is PNGaseF.

5. The method of claim 1, wherein the target protein is cancer antigen 125 or carcinoembryonic antigen.

6. The method of claim 1, wherein the mass spectrometry method comprises a mass spectroscopy selected from the group consisting of matrix-assisted laser desorption/ionization-time-of-flight (MALDI-TOF), electrospray-ionization (ESI), charge detection (CD), Fourier transform-ion cyclotron resonance (FT-ICR), ion-mobility spectrometry (IMS), triple quadrupole, time of flight (TOF), and ion trap.

7. The method of claim 1, wherein the chromatography method comprises a reverse phase separation, a cation exchange separation, an anion exchange separation, an ion pair separation, normal phase separation, an ion mobility separation, a size-exclusion separation, a chiral separation, an affinity separation, a ligand exchange separation, a polar nonionic separation, or any combination thereof.

8. The method of claim 7, wherein the chromatography method is coupled with an UV detection system.

9. The method of claim 1, wherein the predetermined amount of time is between about 10 minutes to 120 minutes.

10. The method of claim 1, wherein the method further comprises heating the sample for a predetermined amount of time prior to subjecting the sample to a chromatography method.

11. The method of claim 10, wherein the heating comprises maintaining the temperature in a range from 30° C. to 80° C.

12. The method of claim 1, wherein the method further comprises buffer exchange and/or desalting processes prior to quantifying the target protein using a mass spectroscopy method.

13. The method of claim 1, wherein the sample is or is derived from a biological fluid selected from the group consisting of blood, urine, spinal fluid, synovial fluid, sputum, semen, saliva, tears, gastric juices and extracts and/or dilutions/solutions thereof.
